(19) United States
(12) Patent Application Publication
(10) Pub. No.: US 2015/0274751 A1
(43) Pub. Date: Oct. 1, 2015

(54) TREATMENT OF SCLERODERMA USING AN INHIBITOR OF CBP/CATENIN

(71) Applicant: PRISM Pharma Co., Ltd., Kanagawa (JP)

(72) Inventors: Hiroyuki Kouji, Kanagawa (JP); Takenao Odagami, Kanagawa (JP)

(73) Assignee: PRISM PHARMA CO., LTD., Kanagawa (JP)

(21) Appl. No.: 14/436,645

(22) PCT Filed: Oct. 21, 2013
(86) PCT No.: PCT/JP2013/079057

§ 371 Date: Apr. 17, 2015

Related U.S. Application Data
(60) Provisional application No. 61/716,080, filed on Oct. 19, 2012.

(51) Int. Cl. C07D 498/04; C07D 487/04

(52) U.S. Cl. CPC C07D 498/04; C07D 487/04

(57) ABSTRACT

The present disclosure relates generally to alpha-helix mimetic structures and specifically to alpha-helix mimetic structures that are inhibitors of β-catenin. The disclosure also relates to applications in the treatment of scleroderma, including diffuse systemic sclerosis and limited systemic sclerosis, and pharmaceutical compositions comprising such alpha helix mimetic β-catenin inhibitors.

### CROSS-REFERENCE TO RELATED APPLICATIONS

This application claims priority to U.S. provisional application 61/716,080, filed Oct. 19, 2012, which is incorporated herein in its entirety.

### BACKGROUND OF THE DISCLOSURE

Wnt/β-catenin signaling is emerging as a forerunner for its critical roles in many facets of human biology. This signaling pathway has roles in embryogenesis, organogenesis, and maintaining tissue and organ homeostasis, and also in pathological conditions such as cancer and other human disorders such as inflammatory disorders and fibrosis. It is also integral in several physiological events such as differentiation, proliferation, survival, oxidative stress, morphogenesis, and others. However, aberrant activation of this pathway is also evident in multiple pathological conditions.

Scleroderma/systemic sclerosis (SSc) is a chronic systemic autoimmune disease (primarily of the skin) characterized by fibrosis (or hardening), vascular alterations, and autoantibodies. There are two major forms. Limited systemic sclerosis/scleroderma involves cutaneous manifestations that mainly affect the hands, arms and face. Diffuse systemic sclerosis/scleroderma is rapidly progressing and affects a large area of the skin and one or more internal organs, frequently the kidneys, esophagus, heart and lungs.

The expression of Wnt-10b was increased in lesional skin biopsy specimens from patients with systemic sclerosis and in those obtained from mice with bleomycin-induced fibrosis. Transgenic mice expressing Wnt-10b showed progressive loss of subcutaneous adipose tissue accompanied by dermal fibrosis, increased collagen deposition, fibroblast activation, and myofibroblast accumulation. Wnt activity correlated with collagen gene expression in these biopsy specimens. Explanted skin fibroblasts from transgenic mice demonstrated persistent Wnt/β-catenin signaling and elevated collagen and α-smooth muscle actin gene expression. Wnt-10b infection of normal fibroblasts and preadipocytes resulted in blockade of adipogenesis and transforming growth factor β (TGFβ)-independent up-regulation of fibrotic gene expression.

SSc is associated with increased Wnt-10b expression in the skin. Ectopic Wnt-10b causes loss of subcutaneous adipose tissue and TGFβ-independent dermal fibrosis in transgenic mice. These findings suggest that Wnt-10b switches differentiation of mesenchymal cells toward myofibroblasts by inducing a fibrogenic transcriptional program while suppressing adipogenesis. Wnt-10b-transgenic mice represent a novel animal model for investigating Wnt signaling in the setting of fibrosis (Wei J et al., Arthritis Rheum. 63(6):1707-17, 2011).

### BRIEF SUMMARY OF THE DISCLOSURE

This disclosure presents methods of treating scleroderma, including limited and diffuse systemic sclerosis, by administration of an inhibitor of β-catenin. This disclosure also provides alpha helix mimetic β-catenin inhibitor compounds, and compositions comprising an inhibitor of β-catenin.

### BRIEF DESCRIPTION OF THE FIGURES

FIG. 1. Summary of dermal fibrosis scores scored as follows: 0=no significant changes; 1=trace to minimal severity; 2=mild severity; 3=moderate severity; 4=marked severity.

### DETAILED DESCRIPTION OF THE DISCLOSURE

Recently, non-peptide compounds have been developed which mimic the secondary structure of reverse-turns found in biologically active proteins or peptides. For example, U.S. Pat. No. 5,440,013 and published PCT Applications Nos. WO94/03494, WO01/00210A1, and WO01/16135A2 each disclose conformationally constrained, non-peptidic compounds, which mimic the three-dimensional structure of reverse-turns. In addition, U.S. Pat. No. 5,929,237 and its continuation-in-part U.S. Pat. No. 6,013,458, disclose conformationally constrained compounds which mimic the secondary structure of reverse-turn regions of biologically active peptides and proteins. In relation to reverse-turn mimetics, conformationally constrained compounds have been disclosed which mimic the secondary structure of alpha-helix regions of biologically active peptide and proteins in WO2007/056513 and WO2007/056593.

This disclosure provides novel compounds, pharmaceutical compositions and methods of treatment for scleroderma. The inventors have determined that inhibiting β-catenin signaling is an effective approach to the treatment of both limited and diffuse systemic sclerosis.

The structures and compounds of the alpha helix mimetic β-catenin inhibitors of this invention are disclosed in WO 2010/044485, WO 2010/128685, WO 2009/148192, US 2011/0092459, each of which is incorporated herein by reference in its entirety. These compounds have now been found to be useful in the treatment of scleroderma.

The preferable structure of the alpha helix mimetic β-catenin inhibitors of this invention have the following formula (I):

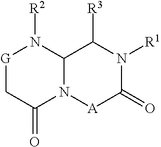

wherein

### A is —CHR⁷—,

wherein

R⁷ is optionally substituted arylalkyl, optionally substituted heteroarylalkyl, optionally substituted cycloalkylalkyl or optionally substituted heterocycloalkylalkyl;

### G is —NH—, —NR⁶—, or —O—

wherein

R⁶ is lower alkyl or lower alkenyl;

### R¹ is —Ra-R¹⁰;

wherein

Ra is optionally substituted lower alkylene and

R¹⁰ is optionally substituted bicyclic fused aryl or optionally substituted bicyclic fused heteroaryl;

### R² is —(CO)—NH—Rb-R²⁰,

wherein

Rb is bond or optionally substituted lower alkylene; and

R²⁰ is optionally substituted aryl or optionally substituted heteroaryl; and

R³ is C₁₋₄ alkyl.
These compounds are especially useful in the prevention and/or treatment of scleroderma.

The more preferable structure of the alpha helix mimetic β-catenin inhibitors of this invention have the following substituents in the above-mentioned formula (I):

### A is —CHR⁷—,

wherein

R⁷ is arylalkyl optionally substituted with hydroxyl or C₁₋₄ alkyl;

### G is —NH—, —NR⁶—, or —O—

wherein

R⁶ is C₁₋₄ alkyl or C₁₋₄ alkenyl;

### R¹ is —Ra-R¹⁰;

wherein

Ra is C₁₋₄ alkylene and

R¹⁰ is bicyclic fused aryl or bicyclic fused heteroaryl, optionally substituted with halogen or amino;

### R² is —(CO)—NH—Rb-R²⁰,

wherein

Rb is bond or C₁₋₄ alkylene; and

R²⁰ is aryl or heteroaryl; and

R³ is C₁₋₄ alkyl.
These compounds are especially useful in the prevention and/or treatment of scleroderma.

The most preferable alpha helix mimetic β-catenin inhibitors of this invention are as follows:
(6S,9S)—N-benzyl-6-(4-hydroxybenzyl)-2,9-dimethyl-8-(naphthalen-1-ylmethyl)-4,7-dioxooctahydro-1H-pyrazino[2,1-c][1,2,4]triazine-1-carboxamide,
(6S,9S)-2-allyl-N-benzyl-6-(4-hydroxybenzyl)-9-methyl-8-(naphthalen-1-ylmethyl)-4,7-dioxooctahydro-1H-pyrazino[2,1-c][1,2,4]triazine-1-carboxamide,
(6S,9S)—N-benzyl-6-(4-hydroxybenzyl)-9-methyl-8-(naphthalen-1-ylmethyl)-4,7-dioxohexahydropyrazino[2,1-c][1,2,4]oxadiazine-1(6H)-carboxamide,
(6S,9S)-8-((2-aminobenzo[d]thiazol-4-yl)methyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide,
(6S,9S)-2-allyl-N-benzyl-6-(4-hydroxybenzyl)-9-methyl-4,7-dioxo-8-(quinolin-8-ylmethyl)octahydro-1H-pyrazino[2,1-c][1,2,4]triazine-1-carboxamide,
4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate,
4-(((6S,9S)-1-(benzylcarbamoyl)-2,9-dimethyl-8-(naphthalen-1-ylmethyl)-4,7-dioxooctahydro-1H-pyrazino[2,1-c][1,2,4]triazin-6-yl)methyl)phenyl dihydrogen phosphate,
sodium 4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl phosphate,
sodium 4-(((6S,9S)-1-(benzylcarbamoyl)-2,9-dimethyl-4,7-dioxo-8-(naphthalen-8-ylmethyl)octahydro-1H-pyrazino[2,1-c][1,2,4]triazin-6-yl)methyl)phenyl phosphate,
(6S,9S)-2-allyl-6-(4-hydroxybenzyl)-9-methyl-4,7-dioxo-N—((R)-1-phenylethyl)-8-(quinolin-8-ylmethyl)octahydro-1H-pyrazino[2,1-c][1,2,4]triazine-1-carboxamide,
(6S,9S)-2-allyl-6-(4-hydroxybenzyl)-9-methyl-4,7-dioxo-N—((S)-1-phenylethyl)-8-(quinolin-8-ylmethyl)octahydro-1H-pyrazino[2,1-c][1,2,4]triazine-1-carboxamide,
(6S,9S)—N-benzyl-6-(4-hydroxy-2,6-dimethylbenzyl)-2,9-dimethyl-4,7-dioxo-8-(quinolin-8-ylmethyl)octahydro-1H-pyrazino[2,1-c][1,2,4]triazine-1-carboxamide,
(6S,9S)-8-(benzo[b]thiophen-3-ylmethyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)-8-(benzo[c][1,2,5]thiadiazol-4-ylmethyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-8-(isoquinolin-5-ylmethyl)-2,9-dimethyl-4,7-dioxooctahydro-1H-pyrazino[2,1-c][1,2,4]triazine-1-carboxamide,
(6S,9S)—N-benzyl-8-((5-chlorothieno[3,2-b]pyridin-3-yl)methyl)-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-2,9-dimethyl-4,7-dioxo-8-(quinoxalin-5-ylmethyl)octahydro-1H-pyrazino[2,1-c][1,2,4]triazine-1-carboxamide, and
(6S,9S)-6-(4-hydroxybenzyl)-2,9-dimethyl-4,7-dioxo-8-(quinolin-8-ylmethyl)-N-(thiophen-2-ylmethyl)octahydro-1H-pyrazino[2,1-c][1,2,4]triazine-1-carboxamide.
   These compounds are especially useful in the prevention and/or treatment of scleroderma.

In a most preferred embodiment, the compound is:
4-(((6S,9S,9aS)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate,

### Or

(6S,9 S,9aS)—N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide.
   These compounds are especially useful in the prevention and/or treatment of scleroderma.

While not wishing to be bound, the effectiveness of these compounds in treating these conditions is based in part on the ability of these compounds to block TCF4/β-catenin transcriptional pathway by inhibiting cyclic AMP response-element binding protein (CBP), thus altering wnt pathway signaling, which has been found to improve outcomes.

A “β-catenin inhibitor” is a substance that can reduce or prevent β-catenin activity. β-catenin activities include translocation to the nucleus, binding with TCF (T cell factor) transcription factors, and coactivating TCF transcription factor-induced transcription of TCF target genes. A “β-catenin inhibitor” can also interfere with the interaction of CBP and β-catenin. Thus, a β-catenin inhibitor inhibits or reduces CBP/β-catenin signaling and activity of the CBP/β-catenin signaling pathway, including reduction of one or more downstream signaling events.

Disclosed herein are alpha helix mimetic β-catenin inhibitor compounds for treatment of scleroderma/systemic sclerosis.

### Diseases

As used herein, “treatment” refers to clinical intervention in an attempt to alter the disease course of the individual or cell being treated, and can be performed during the course of clinical pathology. Therapeutic effects of treatment include without limitation, preventing recurrence of disease, alleviation of symptoms, diminishment of any direct or indirect pathological consequences of the disease, decreasing the rate of disease progression, amelioration or palliation of the disease state, and remission or improved prognosis.

As used herein, the terms “therapeutically effective amount” and “effective amount” are used interchangeably to refer to an amount of a composition of the invention that is sufficient to result in the prevention of the development or onset of scleroderma, or one or more symptoms thereof, to enhance or improve the effect(s) of another therapy, and/or to ameliorate one or more symptoms of scleroderma. For a subject suffering from scleroderma, a preferred therapeutically effective amount is an amount effective to reduce fibrosis, vascular alterations and/or autoantibodies.

A therapeutically effective amount can be administered to a patient in one or more doses sufficient to palliate, ameliorate, stabilize, reverse or slow the progression of the disease, or otherwise reduce the pathological consequences of the disease, or reduce the symptoms of the disease. The amelioration or reduction need not be permanent, but may be for a period of time ranging from at least one hour, at least one day, or at least one week or more. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition, as well as the route of administration, dosage form and regimen and the desired result.

As used herein, the terms “subject” and “patient” are used interchangeably and refer to an animal, preferably a mammal such as a non-primate (e.g., cows, pigs, horses, cats, dogs, rats etc.) and a primate (e.g., monkey and human), and most preferably a human.

The alpha helix mimetic β-catenin inhibitors described herein are useful to prevent or treat disease. Specifically, the disclosure provides for both prophylactic and therapeutic methods of treating a subject at risk of (or susceptible to) scleroderma. Accordingly, the present methods provide for the prevention and/or treatment of scleroderma in a subject by administering an effective amount of the alpha helix mimetic β-catenin inhibitors to a subject in need thereof. For example, a subject can be administered the alpha helix mimetic β-catenin inhibitors in an effort to improve one or more of the factors of a scleroderma condition.

As used herein, “scleroderma” or “systemic sclerosis” is defined as an autoimmune disease characterized by excessive accumulation of connective or scar tissue within the skin or internal organs, accompanied by vascular alterations and presence of autoantibodies. The accumulation of connective/scar tissue in scleroderma is excessive compared to connective tissue levels in normal, healthy skin or organs. This fibrosis is often accompanied by necrosis and/or inflammation. β-catenin signaling plays a role in inducing the over-production and excess accumulation of an extracellular matrix such as collagen.

The cause of scleroderma is unknown. Scleroderma affects the small blood vessels (arterioles) in all organs. First, the endothelial cells of the arteriole die off, along with smooth muscle cells, by apoptosis. They are replaced by collagen and other fibrous material. Inflammatory cells, particularly CD4+ helper T cells, infiltrate the arteriole, and cause further damage. In limited systemic sclerosis, the external skin, such as hands, arms and face, are primarily affected. Pulmonary arterial hypertension may occur in up to one-third of patients and is the most serious complication for this form of scleroderma. In diffuse systemic sclerosis, the skin and internal organs, such as the kidneys, esophagus, heart, and lungs, are affected. This form of systemic sclerosis can be disabling. Both forms of the disease can be life-threatening.

The invention also encompasses methods where the β-catenin inhibitor compound is given in combination therapy. That is, the compound can be used in conjunction with, but separately from, other agents useful in treating scleroderma. In these combination methods, the compound will generally be given in a daily dose of 1-100 mg/kg body weight daily in conjunction with other agents. The other agents generally will be given in the amounts used therapeutically. The specific dosing regime, however, will be determined by a physician using sound medical judgment.

Treatment of scleroderma refers to the administration of a compound or combination described herein to treat a subject suffering from scleroderma. One outcome of the treatment of scleroderma is to reduce formation of excessive connective tissue. Another outcome of the treatment of scleroderma is to reduce inflammation, autoantibodies, and infiltration of immune cells. Still another outcome of the treatment of pulmonary fibrosis is to reduce vascular alterations, such as apoptosis of arteriole endothelial cells and/or smooth muscle cells.

The alpha helix mimetic β-catenin inhibitors described herein can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Any suitable route of administration may be employed for providing a mammal, especially a human, with an effective dose of a compound described herein. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The effective dosage of active ingredient employed may vary depending on the particular compound employed, the mode of administration, the condition being treated and the severity of the condition being treated. Such dosage may be ascertained readily by a person skilled in the art.

When treating or controlling scleroderma and/or other diseases for which compounds described herein are indicated, generally satisfactory results are obtained when the compounds described herein are administered at a daily dosage of from about 0.01 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams. In the case of a 70 kg adult human, the total daily dose will generally be from about 1 milligram to about 500 milligrams. For a particularly potent compound, the dosage for an adult human may be as low as 0.1 mg. In some cases, the daily dose may be as high as 1 gram. The dosage regimen may be adjusted within this range or even outside of this range to provide the optimal therapeutic response.

Oral administration will usually be carried out using tablets or capsules. Examples of doses in tablets and capsules are 0.1 mg, 0.25 mg, 0.5 mg, 1 mg, 2 mg, 5 mg, 10 mg, 15 mg, 20 mg, 25 mg, 30 mg, 40 mg, 50 mg, 100 mg, 200 mg, 250 mg, 300 mg, 400 mg, 500 mg, and 750 mg. Other oral forms may also have the same or similar dosages.

Also described herein are pharmaceutical compositions which comprise a compound described herein and a pharmaceutically acceptable carrier. The pharmaceutical compositions described herein comprise a compound described herein or a pharmaceutically acceptable salt as an active ingredient, as well as a pharmaceutically acceptable carrier and optionally other therapeutic ingredients. A pharmaceutical composition may also comprise a prodrug, or a pharmaceutically acceptable salt thereof, if a prodrug is administered.

The compositions can be suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds described herein can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions as oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharm. Res., 3(6):318 (1986).

Pharmaceutical formulations adapted for topical administration may be formulated as ointments, creams, suspensions, lotions, powders, solutions, pastes, gels, sprays, aerosols, or oils. For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in oil base.

Compounds described herein may also be administered parenterally. Solutions or suspensions of these active compounds can be prepared in water suitably mixed with a surfactant or mixture of surfactants such as hydroxypropylcellulose, polysorbate 80, and mono and diglycerides of medium and long chain fatty acids. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

The present disclosure is further illustrated by the following non-limiting examples.

### Examples

The objective of this study was to assess the effects of the test article Compound A, an alpha helix mimetic β-catenin inhibitor compound, in a murine model of bleomycin-induced dermal fibrosis. Compound A is 4-(((6S,9S,9aS)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate. Negative and positive control articles were phosphate buffered saline (PBS) and dexamethasone (DEX, a known treatment for scleroderma), respectively.

Dose Preparation.

The high-dose (0.5 mg/mL) dosing solution of test article was prepared by mixing 4 mL of test article (stock solution at 20 mg/mL) with 36 mL of PBS. The low-dose (0.05 mg/mL) dosing solution was prepared by mixing 4 mL of the high-dose test article dosing solution (0.5 mg/mL) with 36 mL of PBS. The DEX dosing solution (1 mg/mL) was prepared by mixing 1 mL of the DEX stock solution (3 mg/mL) with 2 mL of PBS. The test and positive control articles were prepared once weekly, aliquoted and stored at 4° C. protected from light.

Forty-five female C3H/HeJ inbred mice (Mus musculus) at ages of approximately 6 weeks were obtained from Jackson Laboratory (Bar Harbor, Me.) for use in the study. Mice were selected for the study since these animals are an accepted species frequently used in pre-clinical evaluation of drugs intended for human use.

Dermal fibrosis was induced locally by six weeks of once-daily subcutaneous injections of bleomycin (0.1 U in 0.1 mL) into a defined area on the back of each mouse. Test and control articles were administered by twice-daily intraperitoneal injection starting from Day 14 (i.e., two weeks after the initiation of bleomycin injection). For mice of Groups 1-4, test/control article were administered (BID, IP) for four weeks, starting from Day 14 (i.e., two weeks after the initial bleomycin injection) administration was performed twice daily. Group 1 received negative control article (PBS) at 200 μL/mouse. Groups 2 and 3 received low- and high-dose test article dosing solutions (0.05 and 0.5 mg/mL Compound A) at doses of 0.5 and 5 mg/kg, respectively. Group 4 received DEX dosing solution (1 mg/mL) at doses of 1 mg/kg. For animals of Groups 2-4, doses were administered at dose volumes of 10 mL/kg, and dose volumes for individual animals were recalculated once weekly based on the most recent body weights. Animals were administered bleomycin and test and control articles in two cohorts, with dosing staggered by one day. Cohort A was composed of the first five animals in each group [x01-x05, where x denoted the study group number (1, 2, 3, or 4)]. Cohort B was composed of the remaining animals (x06-x10). See Table 1.

TABLE 1
Summary of Study Design

| | | Scleroderma | | |
|---|---|---|---|---|
| | | Induction (Day | Treatment | Terminal |
| | Mouse No. | 0-41, SC to | (Days 14-41, | Procedures |
| Group | (Females) | single site, QD) | IP, BID) | (Day 42) |
| 1 | 151-160 | Bleomycin | Vehicle | Body weight |
| | | (0.1 U) | (PBS) | Necropsy |
| 2 | 251-260 | | Compound A | Collect skin at |
| | | | (0.5 mg/kg) | injection site |
| 3 | 351-360 | | Compound A | Histopathology |
| | | | (5.0 mg/kg) | on fixed tissues |
| 4 | 451-460 | | Dexamethasone | |
| | | | (1 mg/kg) | |

Mice were euthanized on Day 42, i.e., following six weeks of bleomycin injection and four weeks of test/control article treatment. A full gross necropsy was performed on each mouse, and any macroscopic abnormality was recorded. At necropsy, skin tissues from the bleomycin injection area and lungs were harvested. Each of these tissues was divided into two halves; one half was fixed in 10% neutral buffered formalin (NBF) for histopathologic analysis at the Testing Facility; the other half was stored at −80° C. for possible additional analysis.

Formalin-fixed skin and lung tissues from each mouse were processed for histopathology. Tissues were dehydrated, embedded in paraffin, and sectioned at 3- to 5-μm thicknesses. Sections were stained with hematoxylin/eosin and Masson's trichrome and cover-slipped. Slides were evaluated for dermal fibrosis and dermal inflammation. Evaluation was performed via light microscopy by a board-certified veterinary pathologist. Dermal fibrosis and inflammation were scored using the industry standard 5-point scoring system as follows: 0=no significant changes; 1=trace to minimal severity; 2=mild severity; 3=moderate severity; 4=marked severity.

Calculations and descriptive statistics (means, standard deviations (SD), and standard errors of the mean (SEM)) were performed using EXCEL (Office 2007; Microsoft, Redmond, Wash.). Where appropriate, inferential statistical analysis was performed using PRISM (Version 5.0; GraphPad Software, Inc., San Diego, Calif.) using One-Way Analysis of Variation (ANOVA) followed by Tukey-Kramer post-test or by non-parametric tests (Kruskal-Wallis followed by Dunn post-test). Histopathology severity scores were analyzed using non-parametric tests. P-values of 0.05 or less (P<0.05) were considered statistically significant. Where P-values are not specified, the results were inspected and differences were considered non-significant (NS; P>0.05).

During the study, decreases (11 to 22%) in mean body weight were seen in all groups. Animals of Group 4 showed the largest decrease in body weight; animals of Group 2 show the smallest decrease in body weight, although the differences among the treatment groups at sacrifice were not statistically significant (P>0.05). These decreases in body weight in animals of Groups 1 to 4 were considered to result from daily dosing with bleomycin. The larger decrease observed in animals of Group 4 presumably reflected the combination of dosing with both bleomycin and DEX.

Results.

Lesions typical of bleomycin-induced dermal fibrosis and inflammation were seen in all groups. Increases in collagen and fibrosis were evident in the dermis and consisted of marked deposition of dense collagen separating the adnexal structures. Multifocal dermal and subdermal inflammation (composed of macrophages and lymphocytes, as well as reduced numbers of neutrophils and eosinophils) were evident along with congestion. Epidermal changes included acanthosis and hyperkeratosis, with proliferation of the keratinocytes. Qualitatively, treatment with the test article appeared to mildly reduce the amount of dermal fibrosis in both test article-treated groups.

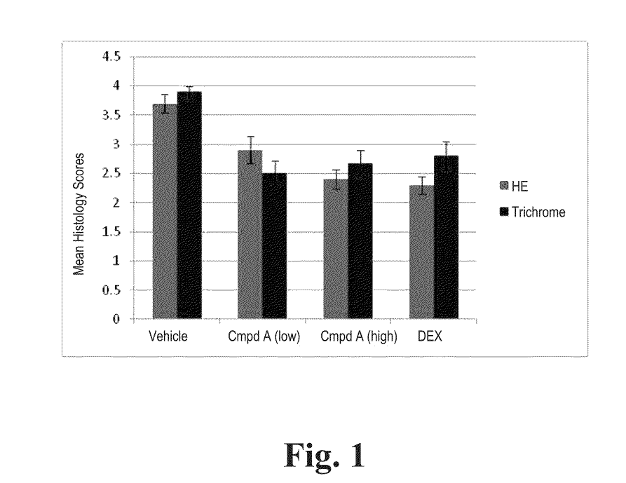

Treatment of bleomycin-injected mice with Compound A did not significantly affect dermal inflammation scores. However, a significant decrease in the dermal sclerosis scores was observed when comparing the vehicle-treated mice to those treated with DEX (P<0.05) or with Compound A at either dose (P<0.01; FIG. 1).

In summary, treatment with Compound A ameliorated dermal fibrosis.

1. An alpha helix mimetic β-catenin inhibitor compound for the treatment of scleroderma, having the following formula (I): 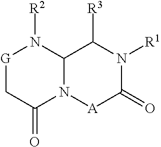
wherein:
A is —CHR⁷—,
wherein R⁷ is hydrogen, optionally substituted alkyl, optionally substituted alkenyl, optionally substituted alkynyl, optionally substituted arylalkyl, optionally substituted heteroarylalkyl, optionally substituted cycloalkylalkyl, optionally substituted heterocycloalkylalkyl, optionally substituted aryl, optionally substituted heteroaryl, optionally substituted cycloalkyl or optionally substituted heterocycloalkyl;
G is —NH—, —NR⁶—, —O—, —CHR⁶— or —C(R⁶)₂—,
wherein R⁶ is independently selected from optionally substituted alkyl, optionally substituted alkenyl and optionally substituted alkynyl;
R¹ is optionally substituted arylalkyl, optionally substituted heteroarylalkyl, optionally substituted cycloalkylalkyl or optionally substituted heterocycloalkylalkyl;
R² is -W²¹-W²²-Rb-R²⁰,
wherein W²¹ is —(CO)— or —(SO₂)—; W²² is bond, —O—, —NH— or optionally substituted lower alkylene; Rb is bond or optionally substituted lower alkylene; and R²⁰ is optionally substituted alkyl, optionally substituted alkenyl, optionally substituted alkynyl, optionally substituted aryl, optionally substituted heteroaryl, optionally substituted cycloalkyl or optionally substituted heterocycloalkyl; and
R³ is optionally substituted alkyl, optionally substituted alkenyl or optionally substituted alkynyl;
or a pharmaceutically acceptable salt thereof.

2. The compound of claim 1, selected from:
(6S,9S)—N-benzyl-6-(4-hydroxybenzyl)-2,9-dimethyl-8-(naphthalen-1-ylmethyl)-4,7-dioxooctahydro-1H-pyrazino[2,1-c][1,2,4]triazine-1-carboxamide,
(6S,9S)-2-allyl-N-benzyl-6-(4-hydroxybenzyl)-9-methyl-8-(naphthalen-1-ylmethyl)-4,7-dioxooctahydro-1H-pyrazino[2,1-c][1,2,4]triazine-1-carboxamide,
(6S,9S)—N-benzyl-6-(4-hydroxybenzyl)-9-methyl-8-(naphthalen-1-ylmethyl)-4,7-dioxohexahydropyrazino[2,1-c][1,2,4]oxadiazine-1(6H)-carboxamide,
(6S,9S)-8-((2-aminobenzo[d]thiazol-4-yl)methyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide,
(6S,9S)-2-allyl-N-benzyl-6-(4-hydroxybenzyl)-9-methyl-4,7-dioxo-8-(quinolin-8-ylmethyl)octahydro-1H-pyrazino[2,1-c][1,2,4]triazine-1-carboxamide,
4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate,
4-(((6S,9S)-1-(benzylcarbamoyl)-2,9-dimethyl-8-(naphthalen-1-ylmethyl)-4,7-dioxooctahydro-1H-pyrazino[2,1-c][1,2,4]triazin-6-yl)methyl)phenyl dihydrogen phosphate,
sodium 4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl phosphate,
sodium 4-(((6S,9S)-1-(benzylcarbamoyl)-2,9-dimethyl-4,7-dioxo-8-(naphthalen-8-ylmethyl)octahydro-1H-pyrazino[2,1-c][1,2,4]triazin-6-yl)methyl)phenyl phosphate,
(6S,9S)-2-allyl-6-(4-hydroxybenzyl)-9-methyl-4,7-dioxo-N—((R)-1-phenylethyl)-8-(quinolin-8-ylmethyl)octahydro-1H-pyrazino[2,1-c][1,2,4]triazine-1-carboxamide,
(6S,9S)-2-allyl-6-(4-hydroxybenzyl)-9-methyl-4,7-dioxo-N—((S)-1-phenylethyl)-8-(quinolin-8-ylmethyl)octahydro-1H-pyrazino[2,1-c][1,2,4]triazine-1-carboxamide,
(6S,9S)—N-benzyl-6-(4-hydroxy-2,6-dimethylbenzyl)-2,9-dimethyl-4,7-dioxo-8-(quinolin-8-ylmethyl)octahydro-1H-pyrazino[2,1-c][1,2,4]triazine-1-carboxamide,
(6S,9S)-8-(benzo[b]thiophen-3-ylmethyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)-8-(benzo[c][1,2,5]thiadiazol-4-ylmethyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-8-(isoquinolin-5-ylmethyl)-2,9-dimethyl-4,7-dioxooctahydro-1H-pyrazino[2,1-c][1,2,4]triazine-1-carboxamide,
(6S,9S)—N-benzyl-8-((5-chlorothieno[3,2-b]pyridin-3-yl)methyl)-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-2,9-dimethyl-4,7-dioxo-8-(quinoxalin-5-ylmethyl)octahydro-1H-pyrazino[2,1-c][1,2,4]triazine-1-carboxamide, and
(6S,9S)-6-(4-hydroxybenzyl)-2,9-dimethyl-4,7-dioxo-8-(quinolin-8-ylmethyl)-N-(thiophen-2-ylmethyl)octahydro-1H-pyrazino[2,1-c][1,2,4]triazine-1-carboxamide.

3. The compound of claim 1, selected from:
4-(((6S,9S,9aS)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate, and
(6S,9S,9aS)—N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide.

4. A pharmaceutical composition comprising the compound of claim 1.

5. A method of treatment for scleroderma, comprising administering an effective amount of the compound of claim 1 to a patient in need thereof.

6. The method of claim 5, wherein the scleroderma is diffuse systemic sclerosis.

7. The method of claim 5, wherein the scleroderma is limited systemic sclerosis.
